(19) United States
(12) Patent Application Publication
(10) Pub. No.: US 2025/0107693 A1
(43) Pub. Date: Apr. 3, 2025

(54) MEDICAL SCOPE DEVICE, STEERING SYSTEM, AND RELATED METHODS

(71) Applicant: Cook Medical Technologies LLC, Bloomington, IN (US)

(72) Inventors: Vincent McHugo, Co. Tipperary (IE); Liam Breen, Co. Tipperary (IE); Jonathan K. Lupton, Thomasville, NC (US); John C. Sigmon, JR., Winston-Salem, NC (US); Shaun D. Gittard, Winston-Salem, NC (US)

(73) Assignee: Cook Medical Technologies LLC, Bloomington, IN (US)

(21) Appl. No.: 18/898,816

(22) Filed: Sep. 27, 2024

Related U.S. Application Data
(60) Provisional application No. 63/586,721, filed on Sep. 29, 2023.

(51) Int. Cl. A61B 1/00; A61B 1/005; A61B 1/012

(52) U.S. Cl. CPC A61B 1/00131; A61B 1/00042; A61B 1/00128; A61B 1/0052; A61B 1/0055; A61B 1/012

(57) ABSTRACT

A scope system, includes an elongate tube having a lumen extending therethrough, the elongate tube including a distal portion; and an accessory channel shaft including a tubular structure with an accessory lumen extending therethrough, wherein the accessory channel shaft is movably disposed at least partially within the lumen of the elongate tube; and a steerable sheath extending at least partially through the accessory lumen, wherein the steerable sheath includes a steerable distal end that is moveable in at least one direction out of an axial direction relative to a distal section of the accessory channel shaft, and an in-line control actuator located at a proximal end of the scope system, wherein operation of the control actuator causes deflection of the steerable distal end relative to the distal section of the accessory channel shaft.

### RELATED APPLICATIONS

This application claims the benefit of the filing date under 35 U.S.C. § 119 (e) of Provisional U.S. Patent Application Ser. No. 63/586,721, filed Sep. 29, 2023, which is hereby incorporated by reference in its entirety.

### BACKGROUND

The statements in this section merely provide background information related to the present disclosure and may not constitute prior art.

The traditional endoscope is a medical device used in a variety of procedures. For example, a physician may insert the endoscope, for example, into a patient's mouth or into another body opening and then manipulate the distal end of the device through the patient's gastrointestinal (GI) tract to perform a particular endoscopic procedure. The physician may then use a variety of instruments during the procedure that are passed through an accessory channel that is located within the outer shaft of the endoscope. As the endoscopy field advances, new endoscopes are being created for specific procedures.

Endoscopes are typically steerable from a proximal-end handle. For example, certain steering features and mechanisms for controlling the distal end of the endoscope are discussed in U.S. Patent Application Pub. No. 2015/0366435, which is hereby incorporated by reference in its entirety. While steerable endoscopes are used with success to treat a variety of issues, existing endoscopes are often challenging to steer when inside the body, particularly when the outer shaft of the endoscope is steered separately from components extending through the accessory channel (e.g., meaning two separate steering systems are needed). As such, the present disclosure presents an improved steering system for use with a variety of endoscopes where a single steering system may control the steering of multiple components in a selective manner.

### BRIEF DESCRIPTION OF THE DRAWINGS

In order that the present disclosure may be well understood, there will now be described various forms thereof, given by way of example, reference being made to the accompanying drawings. The components in the figures are not necessarily to scale. Moreover, in the figures, like-referenced numerals designate corresponding parts throughout the different views.

FIG. 1 is an illustration showing a medical device having a steerable portion in accordance with certain aspects of the present disclosure.

FIG. 2 is an illustration showing the steerable medical device, also having an additional steerable sheath for delivering an endoscopic instrument in accordance with certain aspects of the present disclosure.

FIGS. 3A-3C are illustrations showing various views of the steerable sheath, and particularly a deflectable tip in accordance with certain aspects of the present disclosure.

FIG. 4 is an illustration showing a handle for the steerable sheath in accordance with certain aspects of the present disclosure.

FIGS. 5A-C are illustrations showing various views of a second embodiment of a handle for the steerable sheath in accordance with certain aspects of the present disclosure.

FIGS. 6-7 are illustrations showing various views of a third embodiment of a handle, with a joystick, for the steerable sheath in accordance with certain aspects of the present disclosure.

FIGS. 7A-7C illustrate an alternative joystick embodiment with a steerable disc, shown as part of a scope system in 7A, with more internal components exposed in FIG. 7B, and in longitudinal section (of FIG. 7B) in FIG. 7C

FIGS. 8-9 are illustrations showing a support arm or connection arm for connecting handles of the device in accordance with certain aspects of the present disclosure.

FIGS. 10-12 are illustrations showing a connection embodiment between a support or connection arm and a handle of the device in accordance with certain aspects of the present disclosure.

FIGS. 13-17 are illustrations showing various views of a pre-assembled support or connection arm of the device in accordance with certain aspects of the present disclosure.

FIG. 18 is an illustration showing the support or connection arm during assembly in accordance with certain aspects of the present disclosure.

FIG. 19 shows a diagrammatic transverse section view of a connection arm.

FIGS. 20-22 are illustrations showing various views of a medical device having a joystick-based handle in accordance with certain aspects of the present disclosure.

FIGS. 23-24 are illustrations showing various views of a medical device having a handle for a steerable sheath with a rigid arm, slidable port, and joystick in accordance with certain aspects of the present disclosure.

### DETAILED DESCRIPTION

The following description is merely exemplary in nature and is not intended to limit the present disclosure, application, or uses. It should be understood that throughout the drawings, corresponding reference numerals indicate like or corresponding parts and features.

In adding reference denotations to elements of each drawing, although the same elements are displayed on a different drawing, it should be noted that the same elements have the same denotations. In addition, in describing one aspect of the present disclosure, if it is determined that a detailed description of related well-known configurations or functions blurs the gist of one aspect of the present disclosure, it will be omitted.

In the following discussion, the terms “proximal” and “distal” will be used to describe the opposing axial ends of the device, as well as the axial ends of various component features. The term “proximal” is used in its conventional sense to refer to the end of the device (or component) that is closest to the medical professional during use of the assembly. The term “distal” is used in its conventional sense to refer to the end of the device (or component) that is initially inserted into the patient, or that is closest to the patient during use. The term “longitudinal” will be used to refer to an axial direction that aligns with the proximal-distal axis of the device (or component), for example, when the device is not bent. The terms “radially” and “radial” will be used to refer to elements, surfaces, or assemblies relative to one another that may extend perpendicularly from a longitudinal axis. The terms “circumference,” “circumferentially,” and “circumferential” will be used to elements, surfaces, or assemblies relative to one another encircling or substantially encircling a longitudinal axis at a radius.

The uses of the terms “a” and “an” and “the” and similar references in the context of describing the present disclosure (especially in the context of the following claims) are to be construed to cover both the singular and the plural, unless otherwise indicated herein or clearly contradicted by context. The use of the term “plurality of” is defined by the Applicant in the broadest sense, superseding any other implied definitions or limitations hereinbefore or hereinafter unless expressly asserted by the Applicant to the contrary, to mean a quantity of more than one. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

As used herein, the terms “comprise(s),” “include(s),” “having,” “has,”

“can,” “contain(s),” and variants thereof, are intended to be open-ended transitional phrases, terms, or words that do not preclude the possibility of additional acts or structures. The present description also contemplates other examples “comprising,” “consisting of,” and “consisting essentially of” the elements presented herein, whether explicitly set forth or not.

In describing elements of the present disclosure, the terms 1st, 2nd, first, second, A, B, (a), (b), and the like, may be used herein. These terms are only used to distinguish one element from another element, but do not limit the corresponding elements, irrespective of the nature or order of the corresponding elements.

Unless otherwise defined, all terms used herein, including technical or scientific terms, have the same meanings as those generally understood by those skilled in the art to which the present disclosure pertains. Such terms as those defined in a generally used dictionary are to be interpreted as having meanings equal to the contextual meanings in the relevant field of art.

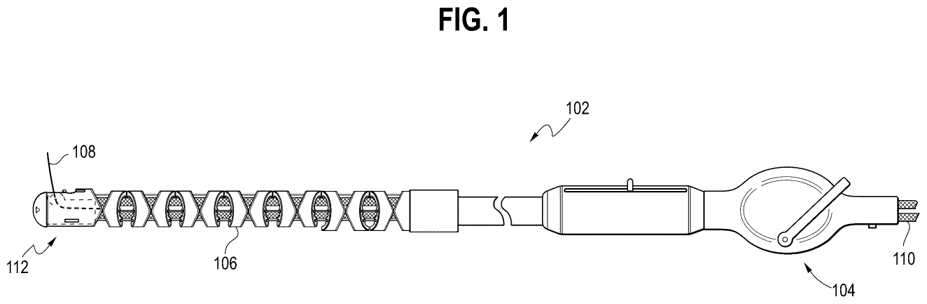

FIG. 1 shows an embodiment of a steerable medical device. The steerable medical device 102 includes a control handle 104 with a steerable outer sheath 106 extending distally therefrom. Without limitation, certain aspects of the steerable medical device 102 may include features discussed in U.S. patent application Ser. No. 16/749,083 (and corresponding Pub. No. 2020/0163534A1), which is hereby incorporated by reference in its entirety. For example, the steerable medical device 102 may generally include an endoscope (of any suitable type), and may include one or more accessory channel shafts 110. The accessory channel shafts may provide lumens or other pathways to a distal end 112 of the device 102 such that accessory components may extend to the device's distal end for performance of desired functions. For example, and without limitation, the accessory channel shafts 110 may receive a steerable device (e.g., a duodenal scope and/or a cholangioscope 108—which is a relatively small-diameter device that is best delivered to a target site via a larger catheter e.g., the outer sheath 106 and/or an accessory channel).

The handle 104 of the device 102 may be located at a proximal end of the steerable medical device 102, and therefore accessible directly by a medical professional. The handle may have any suitable features for steering the device, hereafter referred to as a steering control system 120. Certain aspects of the handle 104 and the control wheels may include features described U.S. patent application Ser. No. 15/655,239 (published as U.S. 2018/028778A1), entitled “STEERABLE MULTILUMEN CATHETER SHAFT,” which is hereby incorporated by reference in its entirety. Other features are described in U.S. Prov. Pat. App. Ser. No. 63/488,220.

In some embodiments, small diameter instruments for use though a small-diameter working channel of the cholangioscope 108 may be used to perform a particular procedure, and such instruments have been used with success, such as biopsy forceps, cytology brushes, stone-removal devices, electrocautery devices, injection needles, and others. However, when such instruments are used with a cholangioscope, they may be limited in size (due to limited working channel size). Thus, the inventors recognize that there would be a benefit to perform the procedures with larger devices that are design with more-optimal parameters (e.g., taking a larger biopsy sample, disintegrate a harder stone faster, removal of a larger stone, etc.). However, when using existing devices (e.g., where the second instrument is side-by-side with a cholangioscope), the control of the location and targeting of these endoscopic instruments is only possible if the forward and reverse direction, as they are not within the cholangioscope's steerable working channel, and as they typically are not steerable on their own.

The present disclosure provides an improved device by incorporating steering into an endoscopic instrument that extends through one or more of the accessory channel shafts 110 or another working channel, particularly via the inclusion of a steerable sheath 202. The steerable sheath 202 may receive an endoscopic instrument through its inner lumen, which may have a diameter (i.e., inner diameter of the sheath) of at least about 2 mm (such as about 2.8 mm in certain advantageous embodiments). An outer diameter of the steerable sheath 202 may be less than about 5 mm (e.g., about 4 mm in certain advantageous embodiments), which allows it to move/slide proximally and distally through the respective working channel of the endoscope.

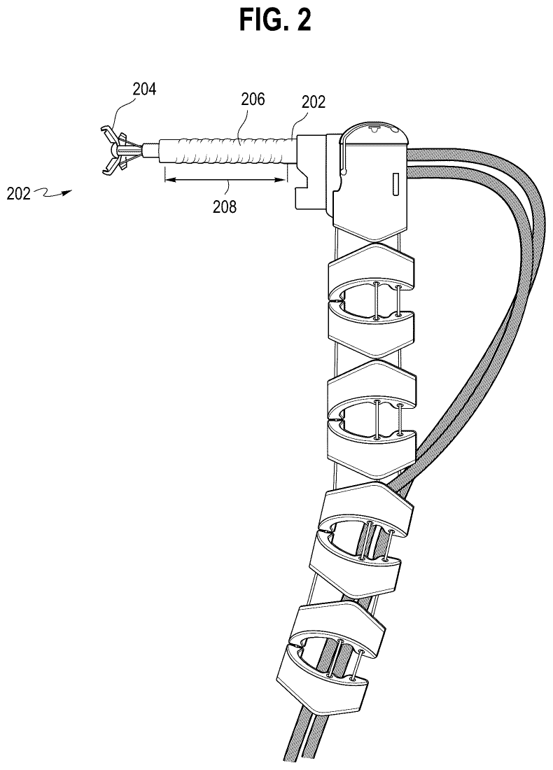

FIG. 2 shows an embodiment having a steerable medical device 102, which may be a delectable endoscope, having the accessory channel shafts 110 in a deflected position at the distal end of the medical device 102. As shown, the steerable sheath 202 is in an extended state, which is a state where the distal end of the steerable sheath 202 extends out of a distal-end-opening of the respective accessory channel shaft 110 such that it is exposed. The steerable sheath 202 receives an endoscopic instrument, in this case a set of biopsy forceps 204, which may be operable by the operating medical professional(s).

As discussed in more detail below, in addition to potential for proximal and distal movement, a distal tip 206 of the steerable sheath 202 may have the ability to deflect in at least one direction. Deflection of the steerable sheath 202 may, as a result, cause control of the position of the biopsy forceps 204 relative to the working channel (e.g., accessory channel shaft 110) of the endoscope with one or more degrees of freedom in addition to proximal and distal linear movement.

Advantageously, the biopsy forceps 204 are controllable not only via the steerability of the accessory channels themselves (which may be advantageous for initial deployment), but also with increased precision at a target patient site within the body. As a further enhancement, the distal tip 206 of the steerable sheath 202 may correspond with an exposed length 208 when the steerable sheath 202 is in the depicted extended position of FIG. 2 (which may be variable and controllable). For example, by increasing the exposed length 208, the potential for deflection relative to the distal terminus of the accessory channel shaft 110 may be increased, and vice versa.

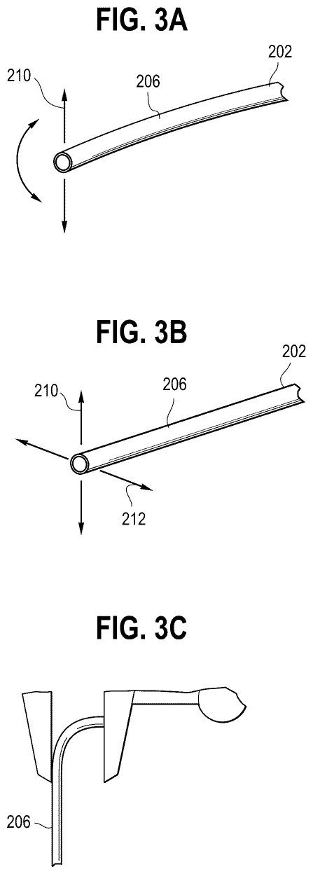

FIGS. 3A and 3B show a variety of potential types of deflection of the distal tip 206 of the steerable sheath 202. As shown in FIG. 3A, the steerable sheath 202 may be steerable in a first direction 210 that is (or at least approaches) perpendicular to the longitudinal direction of the steerable sheath 202, plus is rotatable about its longitudinal axis (and herein, such rotation is included in the meaning of “deflection”). As such, it will be appreciated that by using a combination of horizontal and/or vertical deflection, a user may steer in any direction. In FIG. 3B, the steerable sheath 202 is deflectable in the first direction 210 and also a second direction 212, where the second direction 212 may be perpendicular to the longitudinal axis/direction of the steerable sheath 202 and also may be perpendicular to the first direction 210. These features from FIGS. 3A and 3B may be combined in a single embodiment, and it is contemplated that other types of deflection may also (or alternatively) be included. For illustrative purposes, FIG. 3C shows the steerable sheath 202 in a deflected state (e.g., in the first direction 210) without showing a separate endoscopic instrument.

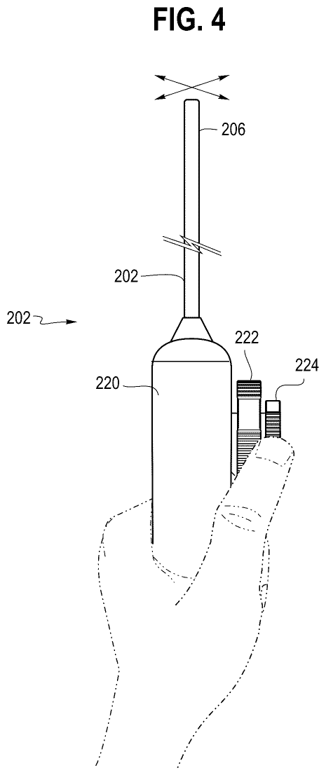

Deflection of the distal tip 206 may be achieved with any suitable device or method. For example, tip deflection may be achieved by way of defection wires running from the distal tip 206 to the proximal end of the medical device, at an accessible position. For example, in certain embodiments and as shown by FIG. 4, the medical device 102 includes a second handle 220 that is dedicated to, and secured to a proximal end of, the steerable sheath 202. In other words, the second handle 220 may be distinct from the handle 104 shown in FIG. 1, which is particularly advantageous where the steerable sheath 202 is insertable into, and removeable from, the accessory channel's proximal end, meaning that it is selectively usable depending on the need of the particular procedure. Alternatively, when the medical device 102 permanently incorporates the steerable sheath 202, the second handle 220 may be secured to the handle 104 to ensure they remain in close proximity.

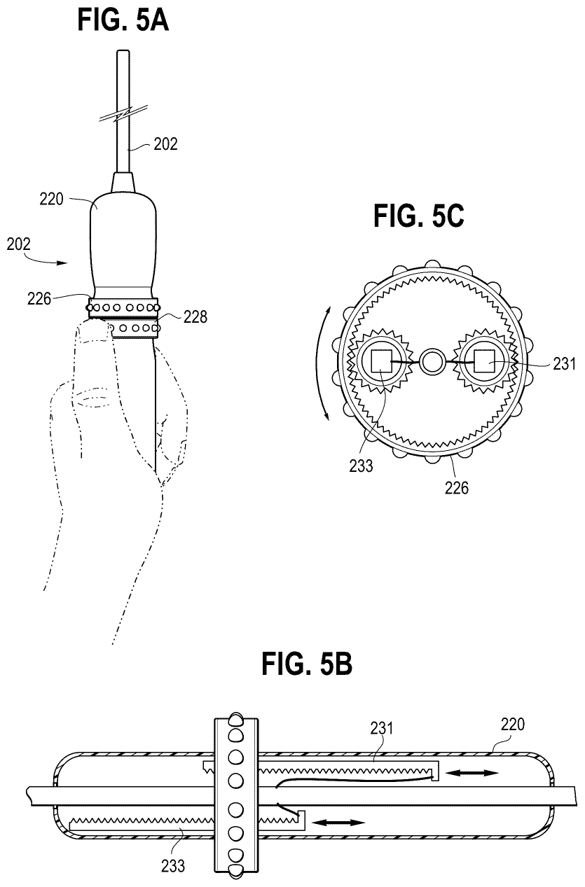

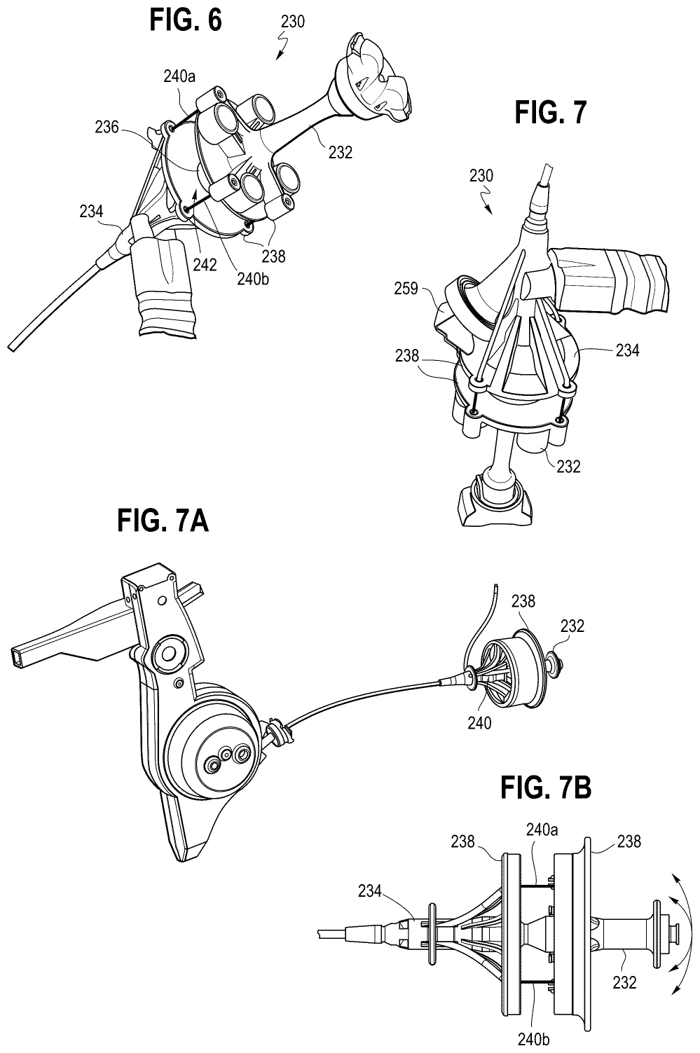

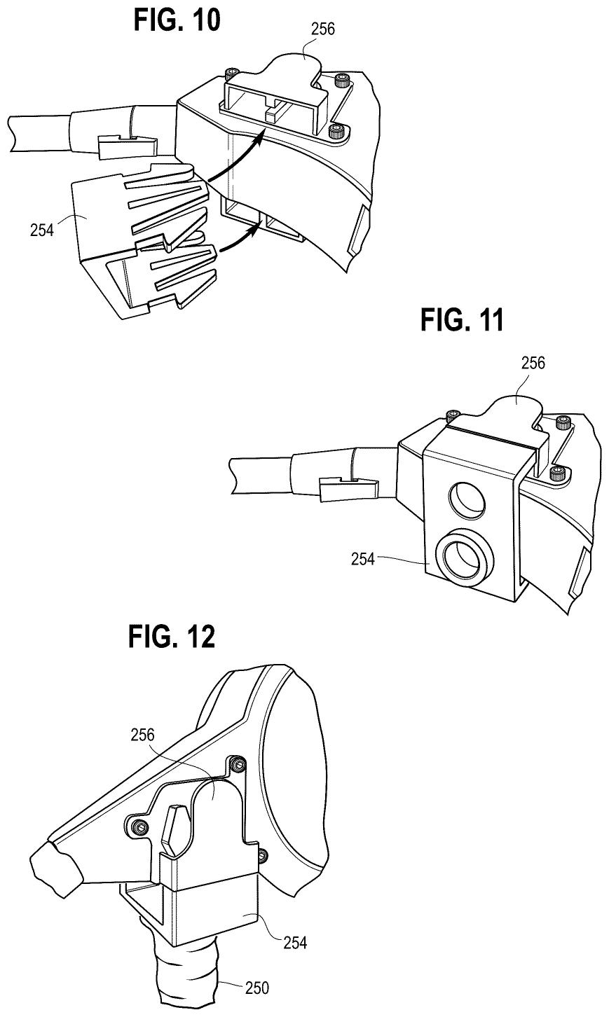

When the second handle 220 is included, it may have any suitable control actuator system for controlling the deflection of the distal tip 206 of the steerable sheath 202. In particular the embodiments of FIGS. 6-22 show in-line control actuator embodiments that differ from other designs (in the prior art and elsewhere in this application) by providing structure for actuating/steering the distal tip 206 by manipulating a structure along an initial/neutral common axis with the sheath 202 and tip 206. Stated differently, an in-line control actuator operates the distal tip by aligned movements instead of translation through a rotary mechanism like traditional control wheels. FIGS. 4-5 show an example of a single-hand control arrangement for the second handle 220, where a first control wheel 222 is rotatable to control a first deflection type (e.g., up/down) and a second control wheel 224 is included to control a second deflection type (e.g., left/right or rotation). Advantageously, the first control wheel and the second control wheel may be co-axial and adjacent to one another such that the thumb of a medical professional can easily switch between types of deflection. Without limitation, the control wheels and other second handle features may have one or more of the aspects discussed in U.S. Prov. Pat. App. No. 63/510,781, where suitable.

Another embodiment of the second handle 220 is shown in FIG. 5, which incorporates a first wheel 226 and a second wheel 228 that spin about the longitudinal axis of the handle. As shown in FIGS. 5A-5C, the second handle 220 may include a rack-and-pinion internal gearing structure that converts rotational movement of the first wheel 226 and the second wheel 228 to linear movement of a first rack 231 and a second rack 233, where the first rack 231 and the second rack 233 are fixed to control wires that extend to the distal tip 206 of the steerable sheath 202.

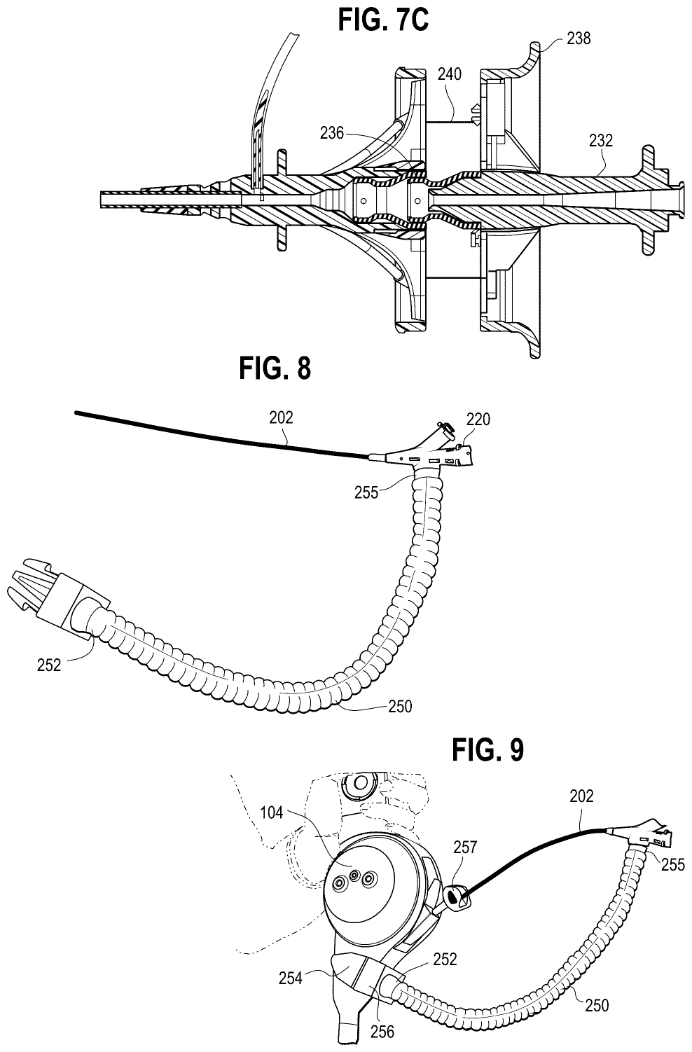

FIGS. 6-7 show an embodiment of the second handle 220 having a joystick 230 for controlling the deflection of the steerable sheath 202. An advantage of the joystick 230 over certain other embodiments is it may be more intuitive and quicker to steer for an operator (as the motion is dynamic, rather than each type of deflection being controlled separately). This embodiment is particularly where a cholangioscopy camera image is in a random orientation to the deflectable sheath tip orientation, meaning that it may be unclear to the operator which direction is which in the camera's image.

While the joystick 230 may have any suitable actuation system, either electronic or mechanical, the depicted embodiment includes a first joystick portion 232 and a second joystick portion 234 connected in a pivotable manner via a ball-and-socket joint 236. Respective first and second plates 238 of the first joystick portion 232 and the second joystick portion 234 may provide a limit to this pivoting action, which may correspond to a limit of movement/deflection of the steerable sheath's distal tip 206. Optionally, the degree of friction provided by the ball-and-socket joint 236 may be selected, e.g. via selection of materials at the contact surfaces, such that it tends to remain in a particular orientation absent external force from the operator, thereby providing a natural brake. Such friction may also increase the precision of operation by being optimally receptive to the operator's motor skills. Alternatively (or in addition), a separate brake device may be included. While not shown, it is contemplated that a yoke mechanism may also be incorporated into the ball and socket to prevent relative axial rotation between the first joystick portion 232 and the second joystick portion 234.

To transfer joystick motion to the distal tip 206, one or more deflection wires 240 may be mechanically engaged with the joystick 230 such that movement of the first joystick portion 232 relative to the second joystick portion 234 causes manipulation of the deflection wires 240. For example, the first deflection wire 240a may extend from the first joystick portion 232, across a central cavity, and to the second joystick portion 234, where the first deflection wire is fixed to the first joystick portion 232 but moveable longitudinally relative to the second joystick portion. Thus, when the joystick moves in the appropriate direction, the first deflection wire will move either proximally or distally relative to the second joystick portion 234, and also more or less of the deflection wire will enter the steerable sheath 202 (meaning its overall length within the steerable sheath 202 changes). This change in deflection wire length is accounted for via tip deflection at the sheath's distal tip 206. A second deflection wire 240b may operate in a similar or identical manner, and additional deflection wires may be placed around the perimeter of the joystick's central cavity 242. In some aspects (such as shown in FIG. 21 below), the joystick 230 may have multiple sections working as a single unit that further enhance the precision of control. This includes that the joystick may be embodied as shown in FIGS. 7A, 7B, and 7C, with upper plate 238 configured as a steerable disc and a shorter protruding joystick portion 232 (which could be omitted in some embodiments).

As mentioned above, it may be desirable to attached the second handle 220 (controlling the steerable sheath) to the first handle 104 (controlling the primary outer sheath 106 and/or accessory channels of the medical device). As shown in FIGS. 8-9, this securement may be provided by a connection arm 250 (also called a “support arm” herein) having a first end 252 fixed to the first handle 104 and a second end 255 fixed to the second handle 220. While some embodiments may call for a rigid connection arm, the depicted connection arm 250 is flexible along its length such that the position of the second handle 220 relative to the first handle 104 may be manipulated for control and comfort. As shown, the steerable sheath 202 extending from the second handle 220 is received in a port 257 leading to an accessory channel shaft 110 of the device. The flexibility of the connection arm 250 may allow for the steerable sheath 202 to be moved further into or out of the port 257 without disconnecting the flexible arm, thereby providing the ability to manually extend/retract the distal tip 206 of the steerable sheath 202 from its respective working channel in the patient's body.

In some embodiments, and referring to FIGS. 9-12, the connection arm 250 may be disconnectable from the first handle 104, which may be desirable when removing the steerable sheath 202 entirely or needing full, unrestricted motion of the second handle 220. While any suitable device may be included, the present embodiment includes a female receiver 254 on the first handle 104 for receiving a buckle 256 secured to the first end of the connection arm 250. The buckle 256 and female receiver 254 are shown as a “double buckle,” meaning each side includes a complete buckle mechanism. The buckle 256 may lock in place in a reliable manner when received, but may be easily removed via pressing the outer buckle buttons inward and pulling the buckle 256 away from the receiver 254. As an alternative, a snap-lock or any other selective attachment may be included.

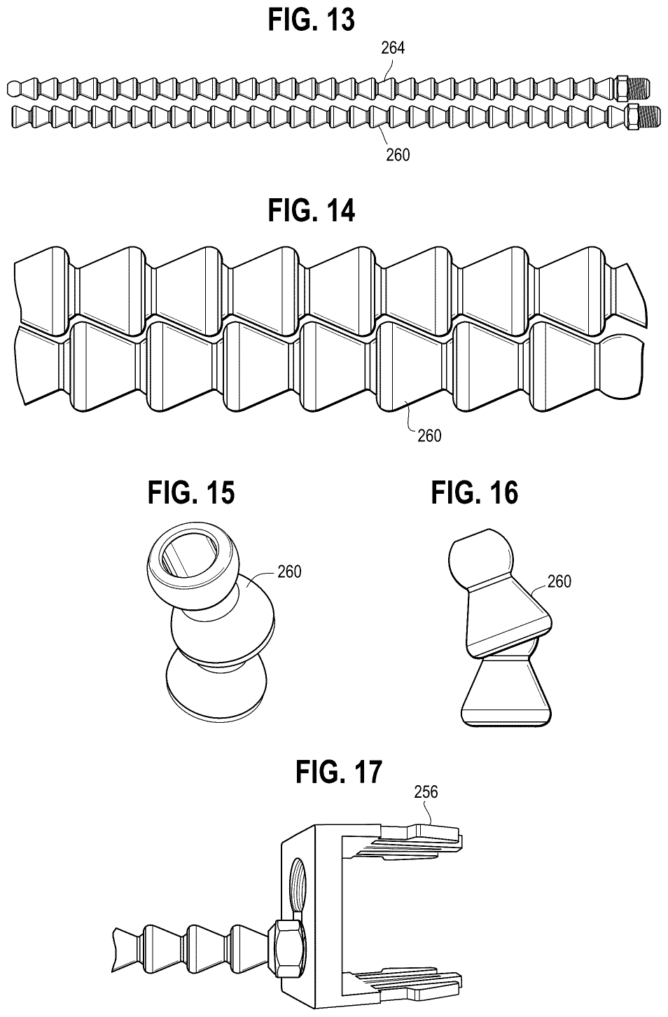

FIGS. 13-17 show various views of the connection arm 250 prior to full assembly. As shown, the securement arm may be made of two hollow tubes 260, each tube 260 having multiple short sections (e.g., 25 each as depicted) that have a partial conical shape, all facing the same direction. A cover of the conical sections maintains their positions relative to each other and provides structure and mechanical integrity. Each conical section may be articulated in any direction (360 degrees) relative to the immediately-adjacent section, thereby allowing a point of “bendability” for the connection arm 250. The material of the cover 264 is stable enough such that the conical sections tend to remain in place relative to each other, absent an outside force. The cumulative result is the connection arm 250 may be bent or otherwise manipulated in nearly all directions, and then maintain that position, upon control by a medical professional.

Using two of the tubes 260 may increase the stability of the connection arm 250. Optionally, the conical sections of the two tubes 260 may face in opposite directions (as shown in FIG. 14), which may be advantageous for saving space and also removing any directional tendency provided by the conical sections arranged in only a single direction. As shown in FIG. 17, the tubes 260 may be secured to the buckle 256 via a keyed socket (and corresponding male keyed portion), but this is optional and other securement devices may be used where suitable.

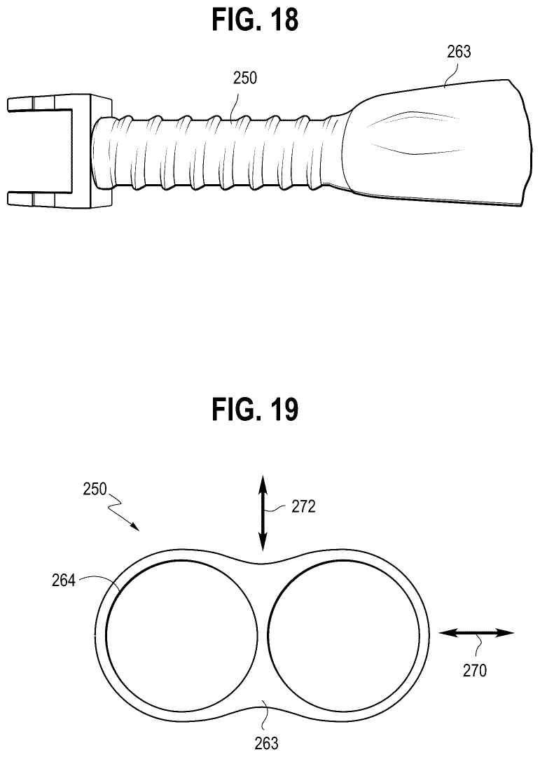

FIG. 18 shows a heat-shrink membrane being installed such that the connection arm 250 appears as a single elongated body. The membrane 263 is optional, but it may be advantageous to ensure the two hollow tubes 260 remain in the same orientations along their length, thereby simplifying the securement arm's control by the medical professional.

FIG. 19 shows a transverse section view of the completed connection arm 250. Another advantage of using two hollow tubes 260 (rather than one) is that stiffness of the connection arm 250 may be different in one direction than the other. In particular, as shown in FIG. 19, stiffness in the first direction 270 is less than stiffness in the second direction 272. This difference in stiffness may be desirable to counteract a weight of the second handle 220, for example, and/or this configuration may be desirable to allow easier movement/flexure (lower stiffness/greater flexibility) up and down as compared to left-right, which can ease movement into and out of the sheath 202.

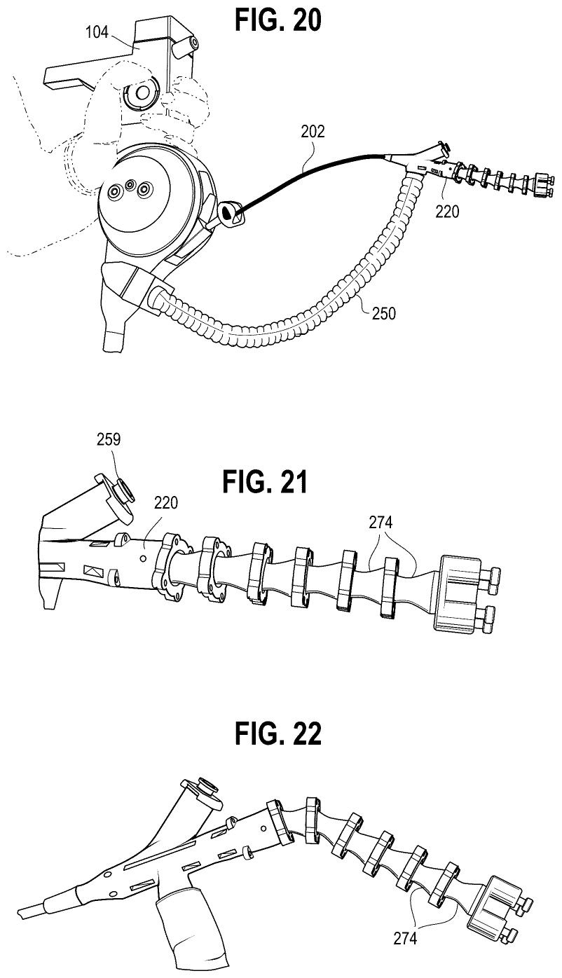

FIGS. 20-22 shows a joystick embodiment, similar to as described above, but with multiple joystick sections 274 (each acting in the manner as described above). The segmented joystick embodiment may be advantageous over a single segment, since it may be optimized with ergonomic touch points, and/or multiple segments may mechanically allow for greater travel distance of the pull wires. As shown in FIG. 21, the second handle 220 may include a second port 259 for receiving an endoscopic instrument, as discussed above. More or fewer entry points may be included. Additional ports can be provided on the handle for wireguide(s), irrigation, contrast, suction, and the like.

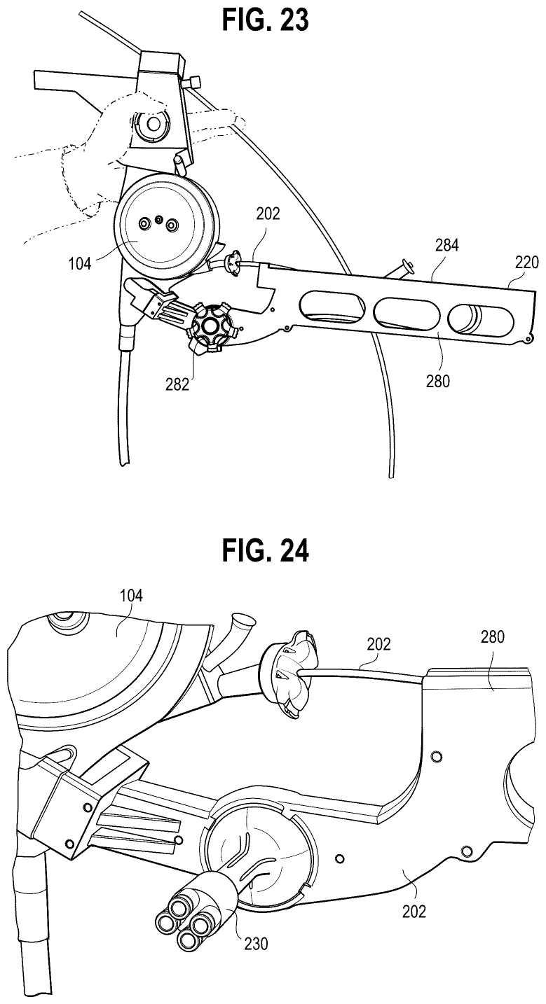

FIGS. 23-24 show another embodiment of the second handle 220, which is depicted as a generally rigid body 280 that is couplable to the first handle 104 via a lockable control wheels 282 (which attachment provide a pivotable connection). A slidable port 257 may be slidable along a track 284 of the rigid arm 280 such that, upon sliding the slidable port 257, and endoscopic instrument may be extended and/or retracted relative to the distal tip 206 of the steerable sheath 202. A joystick 230 may be included, and mechanically coupled to the rigid arm 280 and a set of deflection wires 240, for control of the steerable sheath's deflection.

In some embodiments, radiopaque markers can be provided in the steerable sheath's distal end for fluoroscopic visualization. Additional visual and or tactile marks can be added to the proximal end of the steerable sheath shaft to aid alignment of the steerable sheath to the scope. Color coding can be added to the sheath exterior and a joystick (if included) with the sheath color coding being endoscopically visible; this when matched to the color coding on the joystick would aid in the steering of the sheath tip. In addition, for the low light environment of the endoscopic suite, tactile (brail type) marks can be place on the handle corresponding to visual marks on the sheath distal end, e.g. visual dots on the distal sheath end and raised dots on the handle. In addition, compass cardinal points can be used: N, S, E, W, the meaning of which are widely understood. Also, some cultures have colors associated with the compass cardinal directions which could be used. The use of tactile (Braille type) marks or compass cardinal points can be used in conjunction with color markings to alleviated the issue of color blindness in some users.

While various embodiments have been described, it will be apparent to those of ordinary skill in the art that many more embodiments and implementations are possible. Accordingly, the embodiments described herein are examples, not the only possible embodiments and implementations.

Having described various aspects of the subject matter above, additional disclosure is provided below that may be consistent with the claims originally filed with this disclosure. In describing this additional subject matter, reference may be made to the previously described figures. Any of the following aspects may be combined, where compatible.

In one aspect, embodiments disclosed here include a scope system, with an elongate tube having a lumen extending therethrough, the elongate tube including a distal portion; and an accessory channel shaft including a tubular structure with an accessory lumen extending therethrough, wherein the accessory channel shaft is movably disposed at least partially within the lumen of the elongate tube; and a steerable sheath extending at least partially through the accessory lumen, wherein the steerable sheath includes a steerable distal end that is moveable in at least one direction out of an axial direction relative to a distal section of the accessory channel shaft; and an in-line control actuator located at a proximal end of the scope system, wherein operation of the control actuator causes deflection of the steerable distal end relative to the distal section of the accessory channel shaft. In some embodiments, the control actuator includes a steerable disc that is mechanically connected to one or more deflection wires coupled to the steerable distal end. In some embodiments, the control actuator includes an elongate joystick that is mechanically connected to one or more deflection wires coupled to the steerable distal end.

Some embodiments also include a first handle for controlling a position of the accessory channel shaft and a second handle for controlling the steerable sheath, and may further include the first handle and the second handle being secured via an attachment arm, where—in certain embodiments—the attachment arm includes a pair of hollow tubes extending approximately parallel, and in some embodiments the attachment arm includes a buckle that selectively engages a female receiver fixed to the first handle. In some embodiments with a second handle, the second handle includes a track, the track being configured to receive a sliding port leading to an inner lumen of the steerable sheath. In some embodiments, the steerable sheath is configured to receive an endoscopic instrument through an inner lumen. Some embodiments are constructed such that the steerable sheath enters the accessory channel shaft via a port of a first handle, wherein moving the steerable sheath relative to the port causes the steerable distal end to move relative to a terminus of the accessory channel shaft.

Some embodiments of a scope system include an elongate tube having a lumen extending therethrough, with the elongate tube including a distal portion that is moveable by operation of a first handle; a steerable sheath extending at least partially through an accessory channel shaft of the scope system, wherein the steerable sheath includes a steerable distal end that is moveable relative to a distal section of the accessory channel shaft; a control actuator located at a proximal end of the scope system, wherein operation of the control actuator causes displacement of the steerable distal end relative to the distal section of the accessory channel shaft; and an attachment arm located at a proximal end of the scope system, wherein the attachment arm has a first end secured to the first handle, and wherein the attachment arm has a second end secured to the control actuator such that the control actuator is fixed relative to the first handle absent an intervention force. In some embodiments of that scope system, the control actuator includes a joystick that is mechanically connected to one or more deflection wires coupled to the steerable distal end. In some embodiments, the attachment arm includes a pair of hollow tubes extending approximately parallel. In some embodiments, the attachment arm includes a buckle that selectively engages a female receiver fixed to the first handle. In some embodiments, a second handle includes the control actuator, the second handle comprising a track, the track being configured to receive a sliding port leading to an inner lumen of the steerable sheath. In some embodiments, the steerable sheath is configured to receive an endoscopic instrument through an inner lumen. In some embodiments, the steerable sheath enters the accessory channel shaft via a port of the first handle, and wherein moving the steerable sheath relative to the port causes the steerable distal end to move relative to a terminus of the accessory channel shaft.

Some embodiments of a scope system include an elongate tube having a lumen extending therethrough, the elongate tube including a distal portion; and an accessory channel shaft, the accessory channel shaft including a tubular structure with an accessory lumen extending therethrough, wherein the accessory channel shaft is movably disposed at least partially within the lumen of the elongate tube; and an elongated steerable instrument extending at least partially through a lumen of the accessory channel shaft, wherein the steerable instrument includes a steerable distal end that is moveable in at least one direction out of an axial direction relative to a distal section of the accessory channel; and wherein the movability of the steerable distal end is provided by mechanical communication with a control actuator located at a proximal end of the scope system, wherein operation of the control actuator causes deflection of the steerable distal end relative to the distal section of the accessory channel shaft. In some embodiments, the control actuator includes a steerable disc that is mechanically connected to one or more deflection wires coupled to the steerable distal end, where the one or more deflection wires provide the mechanical communication. And, for some embodiments, the steerable instrument is constructed to enter the accessory channel shaft via a port of a first handle, and wherein moving the steerable instrument relative to the port causes the steerable distal end to move relative to a terminus of the accessory channel shaft.

Those of skill in the art will appreciate that embodiments not expressly illustrated herein may be practiced within the scope of the claims, including that features described herein for different embodiments may be combined with each other and/or with currently-known or future-developed technologies while remaining within the scope of the claims. Although specific terms are employed herein, they are used in a generic and descriptive sense only and not for purposes of limitation unless specifically defined by context, usage, or other explicit designation. It is therefore intended that the foregoing detailed description be regarded as illustrative rather than limiting. And, it should be understood that the following claims, including all equivalents, are intended to define the spirit and scope of this invention. Furthermore, the advantages described above are not necessarily the only advantages of the invention, and it is not necessarily expected that all of the described advantages will be achieved with every embodiment. In the event of any inconsistent disclosure or definition from the present application conflicting with any document incorporated by reference, the disclosure or definition herein shall be deemed to prevail.

We claim:

1. A scope system, comprising:
an elongate tube having a lumen extending therethrough, the elongate tube including a distal portion; and
an accessory channel shaft including a tubular structure with an accessory lumen extending therethrough,
wherein the accessory channel shaft is movably disposed at least partially within the lumen of the elongate tube; and
a steerable sheath extending at least partially through the accessory lumen,
wherein the steerable sheath includes a steerable distal end that is moveable in at least one direction out of an axial direction relative to a distal section of the accessory channel shaft; and
an in-line control actuator located at a proximal end of the scope system, wherein operation of the control actuator causes deflection of the steerable distal end relative to the distal section of the accessory channel shaft.

2. The scope system of claim 1, wherein the control actuator includes a steerable disc that is mechanically connected to one or more deflection wires coupled to the steerable distal end.

3. The scope system of claim 1, wherein the control actuator includes an elongate joystick that is mechanically connected to one or more deflection wires coupled to the steerable distal end.

4. The scope system of claim 1, further comprising a first handle for controlling a position of the accessory channel shaft and a second handle for controlling the steerable sheath.

5. The scope system of claim 4, wherein the first handle and the second handle are secured via an attachment arm.

6. The scope system of claim 5, wherein the attachment arm includes a pair of hollow tubes extending approximately parallel.

7. The scope system of claim 5, wherein the attachment arm includes a buckle that selectively engages a female receiver fixed to the first handle.

8. The scope system of claim 4, wherein the second handle includes a track, the track being configured to receive a sliding port leading to an inner lumen of the steerable sheath.

9. The scope system of claim 1, wherein the steerable sheath is configured to receive an endoscopic instrument through an inner lumen.

10. The scope system of claim 1, wherein the steerable sheath enters the accessory channel shaft via a port of a first handle, and wherein moving the steerable sheath relative to the port causes the steerable distal end to move relative to a terminus of the accessory channel shaft.

11. A scope system, comprising:
an elongate tube having a lumen extending therethrough, the elongate tube including a distal portion that is moveable by operation of a first handle;
a steerable sheath extending at least partially through an accessory channel shaft of the scope system, wherein the steerable sheath includes a steerable distal end that is moveable relative to a distal section of the accessory channel shaft;
a control actuator located at a proximal end of the scope system, wherein operation of the control actuator causes displacement of the steerable distal end relative to the distal section of the accessory channel shaft; and
an attachment arm located at a proximal end of the scope system, wherein the attachment arm has a first end secured to the first handle, and wherein the attachment arm has a second end secured to the control actuator such that the control actuator is fixed relative to the first handle absent an intervention force.

12. The scope system of claim 11, wherein the control actuator includes a joystick that is mechanically connected to one or more deflection wires coupled to the steerable distal end.

13. The scope system of claim 11, wherein the attachment arm includes a pair of hollow tubes extending approximately parallel.

14. The scope system of claim 11, wherein the attachment arm includes a buckle that selectively engages a female receiver fixed to the first handle.

15. The scope system of claim 11, wherein a second handle includes the control actuator, the second handle comprising a track, the track being configured to receive a sliding port leading to an inner lumen of the steerable sheath.

16. The scope system of claim 11, wherein the steerable sheath is configured to receive an endoscopic instrument through an inner lumen.

17. The scope system of claim 11, wherein the steerable sheath enters the accessory channel shaft via a port of the first handle, and wherein moving the steerable sheath relative to the port causes the steerable distal end to move relative to a terminus of the accessory channel shaft.

18. A scope system, comprising:
an elongate tube having a lumen extending therethrough, the elongate tube including a distal portion; and
an accessory channel shaft, the accessory channel shaft including a tubular structure with an accessory lumen extending therethrough, wherein the accessory channel shaft is movably disposed at least partially within the lumen of the elongate tube; and
an elongated steerable instrument extending at least partially through a lumen of the accessory channel shaft,
wherein the steerable instrument includes a steerable distal end that is moveable in at least one direction out of an axial direction relative to a distal section of the accessory channel; and
wherein the movability of the steerable distal end is provided by mechanical communication with a control actuator located at a proximal end of the scope system, wherein operation of the control actuator causes deflection of the steerable distal end relative to the distal section of the accessory channel shaft.

19. The scope system of claim 18, wherein the control actuator includes a steerable disc that is mechanically connected to one or more deflection wires coupled to the steerable distal end, where the one or more deflection wires provide the mechanical communication.

20. The scope system of claim 18, wherein the steerable instrument is constructed to enter the accessory channel shaft via a port of a first handle, and wherein moving the steerable instrument relative to the port causes the steerable distal end to move relative to a terminus of the accessory channel shaft.
